(19) United States
(12) Patent Application Publication
(10) Pub. No.: US 2025/0345317 A1
(43) Pub. Date: Nov. 13, 2025

(54) New pharmaceutical compounds, methods and uses thereof

(71) Applicant: BBIT-THERAPEUTICS, LDA., Porto (PT)

(72) Inventors: Lucília Helena ATAÍDE SARAIVA, Porto (PT); Maria José UMBELINO FERREIRA, Lisboa (PT); Silva Fabião MULHOVO, Maputo (PT); José Luis DA SILVA BORGES COSTA, Porto (PT); Liliana Sofia GOMES RAIMUNDO, Porto (PT); Angela PATERNA, Lisboa (PT); Juliana MEIXEDO CALHEIROS, Porto (PT)

(21) Appl. No.: 18/044,886

(22) PCT Filed: Sep. 10, 2021
(86) PCT No.: PCT/IB2021/058241

§ 371 Date: Mar. 10, 2023

(30) Foreign Application Priority Data
Sep. 10, 2020 (PT) 116714

(51) Int. Cl. A61K 31/439; A61K 45/06; A61P 35/00

(52) U.S. Cl. CPC A61K 31/439; A61K 45/06; A61P 35/00

(57) ABSTRACT

The present disclosure relates to novel compounds according to general Formula I or a pharmaceutically acceptable acid or base addition salts, hydrate, solvate, N-oxide, stereo chemically isomer forms, in particular diastereoisomer, enantiomer or atropisomers, or mixtures thereof, a polymorph or ester thereof. The present disclosure also relates to a pharmaceutical composition comprising a compound or prodrug thereof of Formula I for use in the treatment of conditions influenced by homologous recombination DNA repair pathway and wild-type, mutant and other BRCA1 and/or BRCA2 deficiencies, namely therapy or treatment of cancer.

### TECHNICAL FIELD

The present disclosure relates to novel compounds according to general Formula I or a pharmaceutically acceptable acid or base addition salts, hydrate, solvate, N-oxide, stereochemically isomer forms, in particular diastereoisomer, enantiomer or atropisomers, or mixtures thereof, a polymorph or ester thereof. The present disclosure also relates to a pharmaceutical composition comprising a compound or prodrug thereof of Formula I for use in the treatment of conditions influenced by homologous recombination DNA repair pathway and wild-type, mutant and other BRCA1 and BRCA2 deficiencies, namely therapy or treatment of cancer.

### BACKGROUND

Targeted therapies represent the foundation of personalized cancer treatment, justifying the worldwide investments in this field of anticancer drug development. Targeted therapies differ from conventional chemotherapy by acting on specific molecular targets instead of inducing cell death in nonspecific ways by acting indiscriminately on all rapidly dividing normal and cancerous cells. Thus, as compared to conventional chemotherapy, targeted therapies present lower toxicity to normal cells and reduces undesired side effects on patients. Targeted DNA repair therapies have emerged as a promising strategy to be used as chemo-or radiosensitizers by exploring defects in DNA repair pathways through the concept of synthetic lethality. This approach relies on the presence of a specific gene product that resembles a phenotype induced by a mutation in cancer cells, compatible with viability, that when combined with a second dysfunction in a different gene, results in cell death. Thus, these treatments specifically target cancer cells with minimal side effects on healthy cells.

BRCA1 and BRCA2 (BRCA1/2) tumour suppressor genes have a relevant role both as molecular risk signature and as a prognostic biomarker in several cancer types. Indeed, due to their key role in the maintenance of genomic integrity, a dysfunctional BRCA1/2 activity, either by mutation or low expression levels, is associated with high risk of developing different hereditary and sporadic cancer types, namely breast, ovarian, pancreatic, prostate, laryngeal and fallopian tube cancers. In fact, BRCA1/2 coordinate several cellular processes, with a critical role in DNA repair by homologous recombination. In particular, BRCA1 plays these roles in association with its binding partner, BARD1, which stabilizes and confines BRCA1 to the nucleus, facilitating DNA double strand breaks repair mostly by homologous recombination. As such, disruption of the BRCA1-BARD1 heterodimer results in loss of BRCA1 normal function and decreased expression of BRCA1, BARD1 and other main DNA repair factors.

Despite the relevance of a functional BRCA1 and/or BRCA2 pathway in tumour formation, in established tumours, this is associated with poor prognosis and therapeutic resistance due to a continuous activation of DNA damage repair pathways. In fact, although impaired DNA repair is a major driver for carcinogenesis, a functional repair pathway has been associated with worse prognosis for cancer patients. Consistently, a defective DNA repair pathway may positively influence cancer cells sensitivity to chemo-and radiotherapy, which rely on the induction of DNA damage to induce cell death. Indeed, it was shown that BRCA1-deficient cancers are highly sensitive to double strand breaks-inducing agents such as inter-strand crosslinking agents (e.g. platinum and alkylating agents) and anthracyclines, and other DNA-targeting agents such as poly(ADP-ribose) polymerase inhibitors (PARPi; e.g. olaparib, talazoparib, rucaparib, niraparib). In fact, PARPis were already approved for the treatment of advanced and chemotherapy resistant ovarian cancer and metastatic HER2-negative breast cancer in patients with mutant BRCA1 forms. Despite this, upon dysregulation and overexpression of DNA damage repair factors, cells tend to evade the lethal effects of PARPi. Hence, despite the initial good response, these treatments tend to fail due to the development of resistance. In fact, tumours with heterozygous mutant BRCA1 forms, or loss of heterozigoty, are commonly associated with resistance to PARPis and DNA-damaging agents due to remaining DNA damage repair activity (or to ITS restoration), particularly a functional homologous recombination pathway.

In a clinical setting, PARPi are currently in the forefront of clinical research for BRCA1-deficient cancers. Therefore, more effective DNA repair-inhibiting agents are required, particularly to avoid therapeutic resistance, sensitizing cancer cells to the effect of DNA-damaging agents. In this field, inhibitors of the BRCA1/2 pathway reveal to be promising for resistant and hard-to-treat cancers, inactivating homologous recombination DNA repair. However, despite the relevant role of these proteins in tumorigenesis, effective regulators of their activities are still missing.

These facts are disclosed in order to illustrate the technical problem addressed by the present disclosure.

### GENERAL DESCRIPTION

The present disclosure of compounds of general formula (1), or pharmaceutically acceptable salts, stereoisomer, diastereoisomer, enantiomer, atropisomer, polymorph, for use in medicine or veterinary

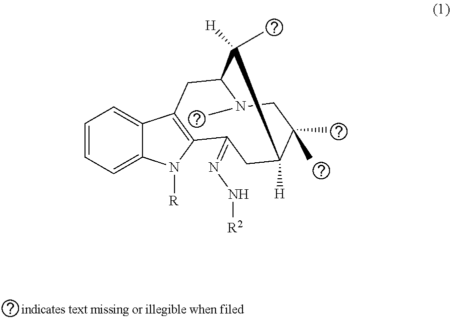

wherein
R¹, R², R³, R⁴, R⁵, R⁶, R⁷, are independently selected from each other;
R¹ is H, alkyl, alkenyl, alkynyl, aryl, aroyl, heteroaryl or heteroarylcarbonyl;
R² is H, alkyl, alkenyl, alkynyl, aryl, aroyl, heteroaryl or heteroarylcarbonyl;
R³ is H or ethyl;
R⁴ is H or ethyl;
R⁵ is COOR⁶, CH₂OR⁶, CONR⁶R⁷ or CH₂NR⁶R⁷;
R⁶ is H, alkyl, alkenyl, alkynyl, aryl or heteroaryl;
R⁷ is H, alkyl, alkenyl, alkynyl, aryl, or heteroaryl, preferably for use in the treatment of conditions associated with a BRCA1 and/or BRCA2-mediated homologous recombination DNA repair pathway, particularly as a disruptor of homologous recombination through inhibition of BRCA1 and or BRCA12.

In an embodiment, the compounds of the present disclosure may be use in the therapy or treatment of a disease that is improved by inhibition of the BRCA1 and or BRCA12 pathway.

In an embodiment, R¹ is selected from: H, alkyl, alkenyl, or alkynyl, preferably R¹ is selected from: H, C1-C6 alkyl, C1-C6 alkenyl or C1-C6 alkynyl.

In an embodiment, R² is selected from: aryl, aroyl, heteroaryl or heteroarylcarbonyl, preferably R² is a heteroaryl.

In an embodiment, R² is a pyridine, more preferably R² is a pyridine with a substituted halogen, more preferably R² is 5-bromopyridin.

In an embodiment, R³ is H and R⁴ is ethyl.

In an embodiment, R³ is ethyl and R⁴ is H.

In an embodiment, R⁵ is selected from COOR6, CONR6R7.

In an embodiment, R⁶ is selected from H, C1-C6 alkyl, C1-C6 alkenyl or C1-C6 alkynyl.

In an embodiment, R⁷ is selected from H, alkyl, alkenyl or alkynyl.

In an embodiment, R⁷ is selected from C1-C6 alkyl, C1-C6 alkenyl or C1-C6 alkynyl.

In an embodiment, R⁵ is COOR⁶ and R⁶ is methyl.

In an embodiment, the compound is methyl(5R,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate or methyl(5,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate.

In an embodiment, the compounds of the present disclosure may be use as an inhibitor of homologous recombination DNA repair through disruption of BRCA1/2 pathway.

In an embodiment, the compounds of the present disclosure may be use as an inhibitor of homologous recombination DNA repair through disruption of BRCA1-BARD1 interaction.

In an embodiment, the compounds of the present disclosure may be use in the prevention, therapy, or treatment of cancer or a tumor.

In an embodiment, the compounds of the present disclosure may be use in the prevention, therapy, or treatment of a solid tumor.

In an embodiment, the compounds of the present disclosure may be use in the prevention, therapy, or treatment of breast cancer.

In an embodiment, the compounds of the present disclosure may be use in the prevention, therapy, or treatment of triple-negative breast cancer.

In an embodiment, the compounds of the present disclosure may be use as a chemoprotectant.

Another aspect of the present disclosure relates to a pharmaceutical composition comprising a pharmaceutically effective carrier and a therapeutically effective amount of the compounds of the present disclosure.

In an embodiment, the pharmaceutical of the present disclosure may further comprise a chemotherapeutic agent.

In an embodiment, the pharmaceutical of the present disclosure may be administered via topical, oral, parenteral or injectable route.

Another aspect of the present disclosure relates to compound of general formula (I), or pharmaceutically acceptable salts, stereoisomer, diastereoisomer, enantiomer, atropisomer, polymorph

wherein
R¹, R², R³, R⁴, R⁵, R⁶, R⁷, are independently selected from each other;
R¹ is H, alkyl, alkenyl, alkynyl, aryl, aroyl, heteroaryl or heteroarylcarbonyl;
R² is H, alkyl, alkenyl, alkynyl, aryl, aroyl, heteroaryl or heteroarylcarbonyl;
R³ is H or ethyl;
R⁴ is H or ethyl;
R⁵ is COOR⁶, CH₂OR⁶, CONR⁶R⁷ or CH₂NR⁶R⁷;
R⁶ is H, alkyl, alkenyl, alkynyl, aryl or heteroaryl;
R⁷ is H, alkyl, alkenyl, alkynyl, aryl, or heteroaryl;
   with the proviso that methyl(5R,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate and methyl(5,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate are excluded, preferably for use in medicine.

In an embodiment, R¹, R², R³, R⁴, R⁵, R⁶, R⁷, are independently selected from each other:
R¹ is H;
R² is heteroaryl;
R³ is H or ethyl;
R⁴ is H or ethyl;
R⁵ is COOR⁶ or CONR⁶R⁷,
R⁶ is H or alkyl;
R⁷ is H, alkyl, alkenyl, alkynyl, aryl, or heteroaryl.

The present disclosure relates to completely different chemical structure of homologous recombination inhibitors from those described so far, the analogs of the compounds.

In an embodiment, the present disclosure relates to a compound (5R, and 5S, 6S,14S,E)-5-ethyl-8-hydrazono-3,14-dimethyl-2,3,4,5,6,7,8,9-octahydro-1H-2,6-methanoazecino [5,4-b]índole(hereinafter COMP) with the ability to inhibit the BRCA1/2 pathway, particularly by disrupting the BRCA1-BARD1 interaction.

In an embodiment, COMP displays potent antitumor activity both in human cancer cells and xenograft mice models. In particular, COMP presents promising antitumor effect against hard-to-treat tumors that still lack effective therapeutic options, namely triple-negative breast cancer and pancreatic cancer, cancers which are frequently associated with poor prognosis and therapeutic resistance. Additionally, COMP has low toxicity in normal cells, and it has not shown toxic side effects in animal models. COMP inactivate homologous recombination through inhibition of the BRCA1/2 pathway, particularly by disruption of the BRCA1-BARD1 interaction, induction of cell cycle arrest, downregulation of DNA repair factors and subsequent enhancement of DNA damage and cell death. COMP also sensitize triple-negative breast cancer and ovarian cancer cells to the effect of cisplatin and olaparib, reducing their effective dose while increasing their apoptotic potential. Importantly, COMP displays promising in vivo antitumor activity in xenograft mice of ovarian cancer cells with no apparent undesirable toxicity. These properties make this compound a superior molecular probe and anticancer drug candidate compared to other DNA-repair inhibiting agents currently available. Most importantly, its ability to inhibit the BRCA1-BARD1 interaction allows a completely new molecular approach that may predict promising clinical applications of COMP for the personalized therapy of a wide range of cancer patients, particularly for those that still lack effective therapeutic options.

The advantages of the compound of the present disclosure include: i) improvement of the anticancer therapy as well as of patient's quality of life by using a more effective and selective chemical agent without the undesirable toxic side effects commonly associated with cancer treatments; ii) the possibility of expanding the population of cancer patients that may benefit from cancer treatments by using a new molecule able to inhibit the BRCA1/2 pathway and consequently the ability of cancer cells to repair DNA damage and grow.

In an embodiment, COMP is used as a chemical probe in the cancer research field to study the involvement of BRCA1/2 in homologous recombination, as well as in other cancer-related processes.

In an embodiment, a formulation containing COMP as active component may be an effective strategy to treat several resistant cancers addicted to DNA repair.

In an embodiment, the compounds of the present disclosure are a new chemical family of inhibitors of homologous recombination, with a completely new mode of action by inhibition of the BRCA1/2 pathway, particularly by disruption of the BRCA1-BARD1 heterodimer.

In an embodiment, the compounds of the present disclosure present a higher antitumor effect than other DNA repair-targeted therapies currently approved for clinical use.

In an embodiment, the compounds of the present disclosure are promising antitumor compositions for hard-to-treat cancers that still lack effective treatment regimens, such as triple-negative breast cancers and pancreatic cancers.

In an embodiment, the compounds of the present disclosure have promising synergistic effects in combination with conventional chemotherapeutic agents and PARPis.

In an embodiment, the compounds of the present disclosure, unlike conventional chemotherapy, has low toxicity in normal cells and no apparent undesirable toxic side effects.

The term “alkyl” is used herein to denote saturated linear, branched, or cyclic alkyl groups.

The term “alkenyl” is used herein to denote an unsaturated straight or branched hydrocarbon having at least one carbon-carbon double bond.

The term “alkynyl” is used herein to denote an unsaturated straight or branched hydrocarbon having at least one carbon-carbon triple bond.

The term “aryl” is used herein is any carbon-based aromatic group including, but not limited to, benzene, naphthalene, etc. The aryl group can be substituted with one or more groups including, but not limited to, alkyl, alkenyl, alkynyl, halide, nitro, amino, hydroxyl, carboxylic acid, carboxylic acid, ketone or alkoxy.

The term “heteroalkyl” is used herein to denote an alkyl group in which at least one carbon atom has been replaced with a heteroatom (e.g., an O, N, or S atom)

The term “aroyl” is used herein to denote an aryl carbonyl group.

The term “heteroaryl” is used herein to denote an aryl group in which said group comprises at least one heteroatom, selected from nitrogen, oxygen and sulfur.

The term “heteroarylcarbonyl” is used herein to denote an heteroaryl carbonyl group.

### BRIEF DESCRIPTION OF THE DRAWINGS

The following figures provide preferred embodiments for illustrating the disclosure and should not be seen as limiting the scope of invention.

FIG. 1 shows the general structure of (5R, and 5S, 6S,14S,E)-5-ethyl-8-hydrazono-3, 14-dimethyl-2,3,4,5,6,7,8,9-octahydro-1H-2,6-methanoazecino[5,4-b]indole (COMP).

FIG. 2 illustrates the growth inhibitory effect of COMP in a panel of human immortalized normal (MCF10a and HFF-1) and cancer cell lines (T47D, MCF-7, MDA-MB-231, MDA-MB-468, SK-BR-3 and HCC193, OCVAR, SKOV-3, SK-BR-3, IGROV-1 and HeLa, PANC-1, MIAPACA, SHSY-5Y, NCI-H460, VCaP, A375, SK-MEL-5 and SF-208. IC50 values were determined by the SRB assay after 48 hours of treatment with COMP. Data are mean±SEM (n=5).

FIG. 3 illustrates the significant differences between concentration-response curves for the growth inhibitory effect of COMP on ovarian and triple-negative breast cancer cells as compared to normal (MCF10a and HFF1) cells, determined by the SRB assay after 48 hours of treatment. Data are mean±SEM (n=5); growth obtained with DMSO was set as 100%; values obtained from normal cells are significantly different from cancer cells: *** P<0.001 (one-way ANOVA followed by Dunnett's test).

FIG. 4 illustrates the effect of COMP on (A-B) colony formation of cancer cells after 8 days (MDA-MB-231 and IGROV-1) and 16 days (HCC1937) of treatment. In FIG. 4A, representative experiments are shown. In FIG. 4B, quantification of colony formation; growth obtained with DMSO was set as 100%; data are mean±SEM (n=5); * P<0.05 and *** P<0.001 significantly different from DMSO (two-way ANOVA followed by Sidak's test).

FIG. 5 illustrates the effect of COMP on (A-B) HCC1937 mammosphere formation after 72 hours of treatment with COMP; treatment was performed at seeding time of HCC1937 cells or at (C-D) three-day-old HCC1937 mammospheres for up to 11 days of treatment. FIG. 5A and FIG. 5C are representative images (scale bar=50 μm, 100× magnification). FIG. 5B and FIG. 5D shows the mammosphere area at the end of treatment; data are mean±SEM (n±5); * P<0.05 and *** P<0.001significantly different from DMSO (student's t-test).

FIG. 6 illustrates the effect of 12 μM COMP on the (A-B) expression of key proteins involved in homologous recombination, proliferation and chemoresistance in triple-negative breast cancer and ovarian cancer cells after 48 hours of treatment. FIG. 6A shows representative immunoblots detected by western blot analysis; GAPDH was used as loading control. FIG. 6B shows quantification of protein expression levels relative to DMSO; data are mean±SEM (n=3); * P<0.05 significantly different from DMSO (student's t-test).

FIG. 7 illustrates the effect of 6 and 12 μM COMP on (FIG. 7A) cell cycle progression, (FIG. 7B) apoptosis and (FIG. 7C) ROS generation, in triple-negative breast cancer and ovarian cancer cells after 48 hours of treatment; data are mean±SEM (n=5); values are significantly different from DMSO: * P<0.05, ** P<0.01, *P<0.001 (two-way ANOVA followed by Dunnett's test). Cell cycle phases were analysed by flow cytometry using propidium iodide (PI) and quantified using the FlowJo software. Apoptosis and ROS were analysed by flow cytometry using FITC-Annexin V/PI and 2′,7′-dichlorodihydrofluorescein diacetate (H2DCFDA) respectively.

FIG. 8 illustrates the effect of 6 and 12 μM COMP on triple-negative breast cancer and ovarian cancer cells' DNA damage after 48 hours of treatment, measured by comet assay. FIG. 8A are representative images (scale bar=20 μm; 200× magnification). FIG. 8B shows the quantification of tail DNA percentage (percentage of comet-positive cells with more than 5% of DNA in the tail). FIG. 8C shows quantification of tail moment (product of the tail length and % of DNA in the tail) using TriTek Comet Score imaging software V2.0; data are mean±SEM (n=3-4; 200 cells per sample); * P<0.05 significantly different from DMSO (two-way ANOVA followed by Dunnett's test).

FIG. 9 illustrates the effect of 2 and 6 μM COMP after 48 hours of treatment on homologous recombination activity in MCF7 DR-GFP cells using the chromosomal DR-GFP assay. After 72 hours of double strand breaks induction, MCF7 DR-GFP cells were analysed by flow cytometry to quantify the percentage of GFP-positive cells. Data are mean±SEM (n=4); * P<0.05 significantly different from DMSO: (one-way ANOVA followed by Dunnett's test).

FIG. 10 illustrates the effect of 12 μM COMP on (A) γH2AX expression levels and on (FIG. 10B) γH2AX and RAD51 foci formation, and BRCA1 foci formation and cellular localization after 48 hours of treatment. FIG. 10A shows immunoblots of one of three independent experiments conducted; GAPDH was used as loading control. FIG. 10B are representative images generated using Fiji software (scale bar=100 μm; 400 × magnification). Quantification of γH2AX (FIG. 10C), RAD51 (FIG. 10D) and BRCA1 (FIG. 10E) foci formation; data are mean±SEM (n=3; 100 cells per sample); *P<0.05 significantly different from DMSO (student's t-test).

FIG. 11 illustrates the disruption of the BRCA1-BARD1 interaction by COMP in triple-negative breast cancer and ovarian cancer cells. (FIGS. 11A-D) Co-IP was performed in MDA-MB-231 (FIG. 11A), HCC1937 (FIG. 11B) and IGROV-1 (FIG. 11C) cells treated with 12 and 20 μM COMP for 18 hours (in MDA-MB-231 and HCC1937 cells) and 24 hours (in IGROV-1 cells). Assay was performed using the Pierce classic magnetic IP and Kit followed by western blot detection. In FIGS. 11A-C, representative immunoblots are shown; whole-cell lysate (Input). FIG. 11D shows quantification of BARD1 relative to DMSO (set as 1); BRCA1 from IP was used as loading control; data are mean±SEM (n=3); * P<0.05 significantly different from DMSO (student's t-test).

FIG. 12 illustrates the prevention of HCC1937 cells migration by COMP. Confluent cells were treated with 1.9 μM COMP or DMSO and observed at different time-points in the wound healing assay (scale bar=50 μm and magnification=100×).

FIG. 13 illustrates that COMP sensitizes triple-negative breast cancer and ovarian cancer cells to the effect of cisplatin (CDDP) and olaparib. Cells were treated with a concentration range of CDDP and olaparib alone and in combination with a single concentration of COMP (with no significant effect on cells growth) in MDA-MB-231 (FIG. 13A), HCC1937 (FIG. 13B) and IGROV-1 (FIG. 13C). Cell proliferation was measured by SRB assay after 48 hours of treatment; growth obtained with control (DMSO) was set as 100%. Data are mean±SEM (n=6); * P<0.05 significantly different from chemotherapeutic drugs alone (two-way ANOVA followed by Sidak's test). Combination index (CI) and dose-reduction index (DRI) for each combined treatment were calculated using CompuSyn software (CI<1, synergy; 1<CI<1.1, addictive effect; CI>1.1, antagonism); data were calculated using a mean value effect of six independent experiments.

FIG. 14 illustrates the in vivo antitumor activity of COMP. C57BL/6-Rag2−/−IL2rg−/− xenograft mice, carrying IGROV-1 cells, were treated with vehicle (control), or 2 mg/kg of COMP or 50 mg/kg of olaparib by intraperitoneal injection three times per week (seven administrations in total). The treatment was initiated when palpable tumors were established (^{˜}100 mm3). FIG. 14A shows tumor volume curves of xenograft mice treated with COMP, olaparib or vehicle; relative tumor volumes were plotted for control and treated groups by dividing the tumor volume for each data point by starting tumor volume; values significantly different from vehicle: * P<0.0001 (two-way ANOVA with Turkey's multiple comparison test). FIG. 14B shows mice body weight measured during treatment under each condition, no significant differences between vehicle and COMP-treated mice weight (p>0.05; unpaired Student's t-test) was observed. FIG. 14C shows weight of spleen, liver, heart and kidneys of animals treated with COMP or vehicle. In FIGS. 14A-C, data are mean±SEM, n=13 animals. In FIG. 14B and FIG. 14C, values not significantly different from vehicle: P>0.05 (two-way ANOVA with Turkey's multiple comparison test).

### DETAILED DESCRIPTION

The present disclosure relates to compounds which inhibit homologous recombination DNA repair through inactivation of BRCA-1 and or BRCA1-2 pathway, particularly by disruption of BRCA1-BARD1 interaction and disruption of BRCA2 activity.

In an embodiment, the compound of the present disclosure is (5R, and 5S, 6S,14S,E)-5-ethyl-8-hydrazono-3,14-dimethyl-2,3,4,5,6,7,8,9-octahydro-1H-2,6-methanoazecino [5,4-b]indole (COMP) with general formula (1),

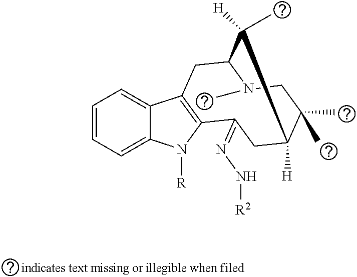

wherein:
R¹ is selected from H, alkyl, alkenyl, alkynyl, aryl, aroyl, heteroaryl or heteroarylcarbonyl;
R² is selected from H, alkyl, alkenyl, alkynyl, aryl, aroyl, heteroaryl or heteroarylcarbonyl;
R³ is selected from H or ethyl;
R⁴ is selected from H or ethyl;
R⁵ is selected from COOR⁶, CH₂OR⁶, CONR⁶R⁷, CH₂NR⁶R⁷, where
R⁶ is selected from H, alkyl, alkenyl, alkynyl, aryl or heteroaryl;
R⁷ is selected from H, alkyl, alkenyl, alkynyl, aryl, or heteroaryl; for use as inhibitor of BRCA1/2-mediated homologous recombination DNA repair.

In an embodiment, the 5R epimer is represented by R³ is H and R⁴ is ethyl.

In an embodiment, the5S epimer is represented by R³ is ethyl and R⁴ is H.

In an embodiment, the analogs of compounds (5R, and 5S, 6S,14S,E)-5-ethyl-8-hydrazono-3, 14-dimethyl-2,3,4,5,6,7,8,9-octahydro-1H-2,6-methanoazecino[5,4-b]indole can be used as molecular probe in DNA repair pathway and BRCA1/2 research field, as chemopreventive, suppressing tumor formation, or as chemotherapeutic, suppressing tumor progression and dissemination of several cancer types, including breast, ovarian, endocervical, pancreatic, prostate, skin, lung, glioblastoma and neuroblastoma. This compound, or its pharmaceutically acceptable salt, represents a completely new chemical family of DNA repair-inhibiting agents, particularly of homologous recombination repair pathway, with high potency as anticancer agent. Most interestingly, it presents a new mechanism of action of BRCA1 inhibition, through disruption of the BRCA1-BARD1 interaction, with high selectivity towards cancer cells. Additionally, the presently disclosed compound has no apparent undesirable toxic side effects. Altogether, this technology will allow improving anticancer therapy and patient's quality of life, and to expand the population of cancer patients that may benefit from cancer treatments, particularly for those that still lack effective treatments.

In a preferred embodiment, the methyl (5R, and 5S, 6S,14S,E)-5-ethyl-8-hydrazono-3,14-dimethyl-2,3,4,5,6,7,8,9-octahydro-1H-2,6-methanoazecino[5,4-b]indole is used for the treatment of conditions associated with BRCA1/2-mediated DNA repair, particularly homologous DNA recombination.

In an embodiment, the present disclosure also relates to pharmaceutical compositions comprising therapeutically effective amount of the compound of the present disclosure and further comprises a pharmaceutically effective carrier.

In an embodiment, the pharmaceutical compositions comprising the compound of the present disclosure further comprise a chemotherapeutic agent.

In an embodiment, the compound of the present disclosure, or the pharmaceutical compositions comprising the compound of present disclosure can also be used as chemoprotectants.

In an embodiment, the compound of the present disclosure, or the pharmaceutical compositions comprising the compound of the present disclosure are administered via topical, oral, parenteral or injectable route.

In an embodiment, preparation of COMP “Methyl(5R,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate” was prepared by derivatization of the monoterpene indole alkaloid dregamine, a natural product obtained from the alkaloid fraction of the African medicinal t Tabernaemontana elegans (Apocynaceae), as outlined in Scheme 1.

In an embodiment, Dregamine (1 mmol) was dissolved in MeOH (3 mL) with 5-bromo-2-hydrazinopyridine (3 mmol) and a catalytic amount of acetic acid. The mixture was stirred under reflux for 24 hours. The reaction mixture was extracted with EtOAc and the organic layers were combined and dried (Na₂SO₄). The solvent was removed under vacuum at 40° C. and the residue obtained was purified by column chromatography (aluminium oxide, n-hexane/CH2Cl2 1:0 to 1:1) to obtain compound 1.
IR (NaCl) vmax 3601, 1728, 1637 cm-1;
HRTOFESIMS m/z 546.1473 [M+Na]+(calcd for C26H30BrN5O2Na, 546.1481); 1H NMR (400 MHZ, CDCl3) δ 8.88 (1H, s, N—H), 8.15 (1H, d, J=2.0 Hz, H-6′), 7.64 (1H, dd, J=8.9, 2.1 Hz, H-4′), 7.57 (1H, d, J=7.9 Hz, H-9), 7.27 (1H, m, H-12), 7.22 (1H, m, H-11), 7.18 (1H, d, J=8.7 Hz, H-3′) 7.09 (1H, t, J=7.5 Hz, H-10), 3.96 (1H, td, J=8.1, 3.0 Hz, H-5), 3.20 (1H, dd, J=14.6, 8.3 Hz, H-6a), 3.01 (1H, dd, J=14.6, 8.3 Hz, H-6b), 2.75 (4H, m, H-14, H-15, H-16), 2.60 (3H, s, —COOMe), 2.54 (3H, s, N—Me), 2.50 (1H, m, H-21a), 2.43 (1H, m, H-21b), 1.81 (1H, m, H-20), 1.41 (2H, m, H-19), 1.02 (3H, t, J=7.3 Hz, H-18) ppm.

13C NMR (101 MHZ, CDCl3) δ 171.1 (—COOMe), 155.6 (C-3), 148.1 (C-6′), 142.1 (C-2′), 140.5 (C-4′), 135.8 (C-13), 133.0 (C-2), 129.8 (C-8), 124.2 (C-11), 119.6 (C-10), 118.8 (C-9), 114.2 (C-7), 110.5 (C-12), 109.9 (C-5′), 109.2 (C-3′), 58.0 (C-5), 50.4 (—COOMe), 48.9 (C-16), 48.2 (C-21), 43.3 (C-20), 42.5 (N-Me), 32.1 (C-14), 31.2 (C-15), 24.0 (C-19), 19.8 (C-6), 12.2 (C-18) ppm.

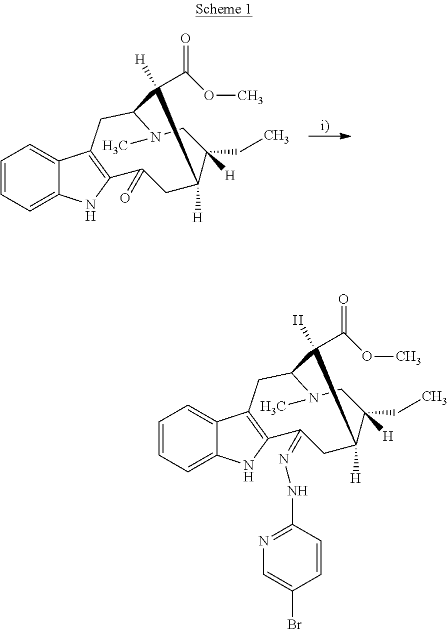

Reagents and conditions: i) 5-bromo-2-hydrazinopyridine (3 equiv.) in MeOH, acetic acid (cat.), reflux, 24 h

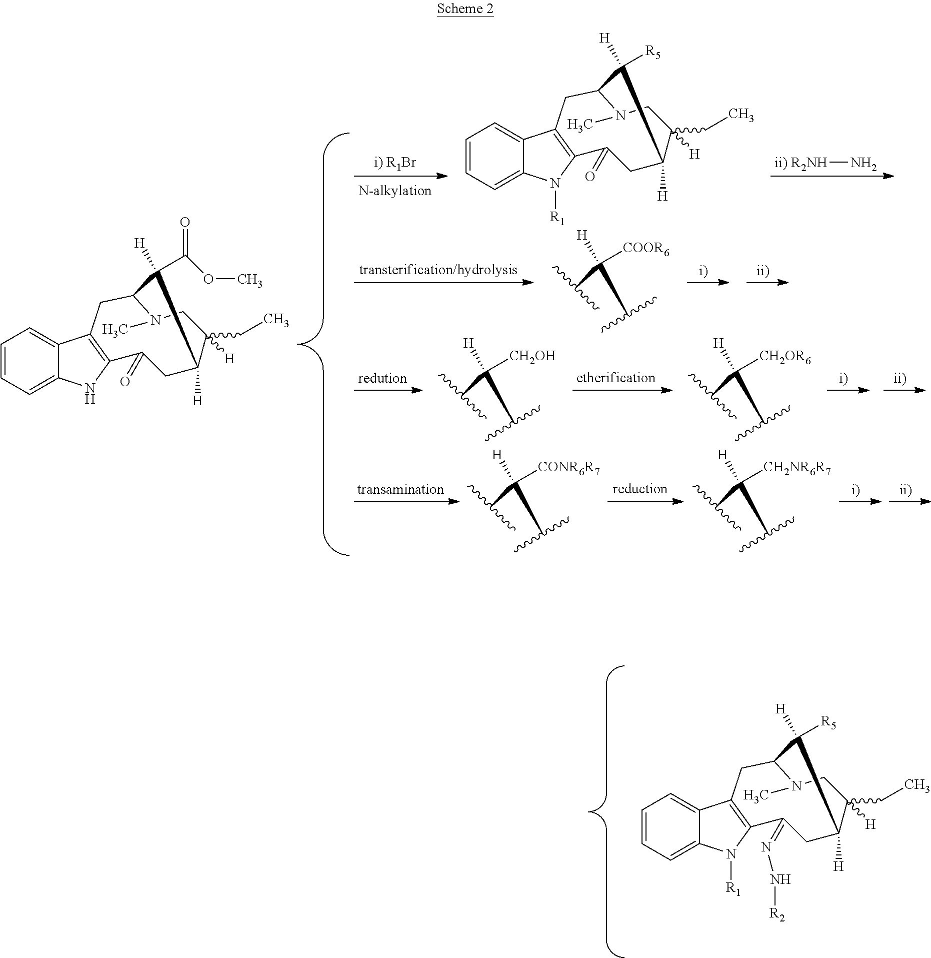

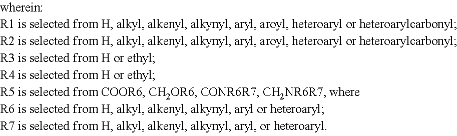

TABLE 1
Structure of compounds.

| | | Molecular | |
|---|---|---|---|
| Compound | | weight | |
| D | C₂₁H₂₆N₂O₃ | 3542.44 | 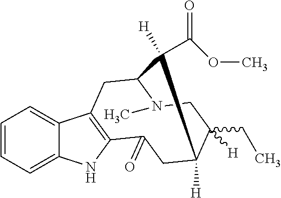 |
| T | C₂₁H₂₆N₂O₃ | 354.44 | 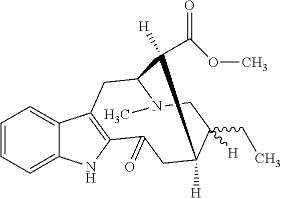 |
| DH1 | C₂₁H₂₈N₄O₂ | 368.47 | 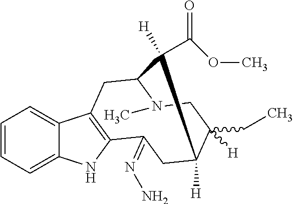 |
| DH2 | C₂₇H₃₂N₄O₂ | 444.57 | 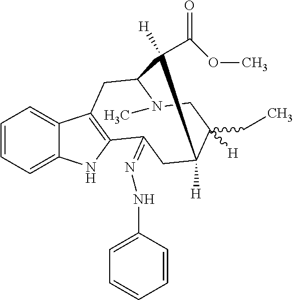 |
| DH3 | C₂₄H₃₀N₆O₂ | 446.54 | 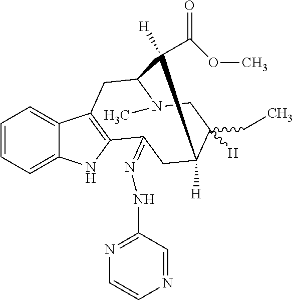 |
| DS2 | C₃₀H₃₆N4O₅ | 532.63 | 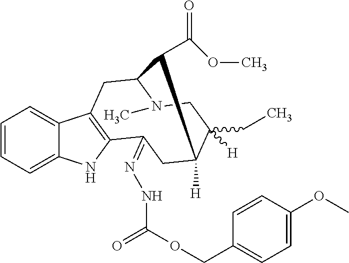 |
| DS3 | C₂₈H₃₄N₆O₂S | 518.67 | 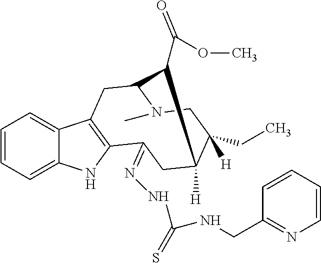 [SMILES: CC[C@H]1CN(C)C2Cc3c([nH]c4ccccc34)/C(=N/NC(=S)NCc3ccccn3)C[C@H]1[C@@H]2C(=O)OC] |
| TH2 | C₂₇H₃₂N₄O₂ | 444.57 | 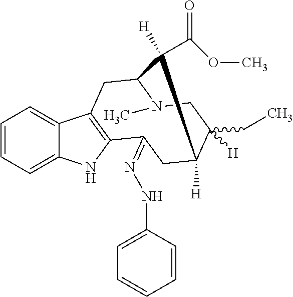 |
| TH4 | C₂₆H₃₀BrN₅O₅ | 524.45 | 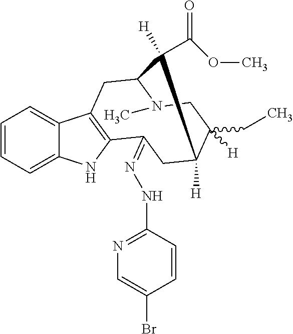 |
| DOH | C₂₁H₂₈N₂O₃ | 356.46 | 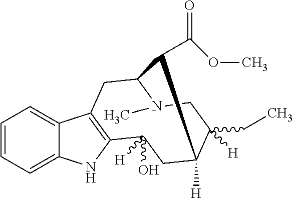 |
| DTHIO | C₂₁H₂₆N₂O₂S | 370.51 | 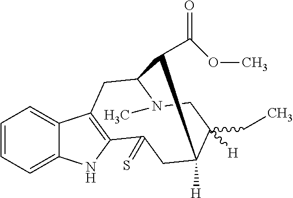 |
| DE1 | C₂₅H₃₂N₂O₄ | 424.24 | 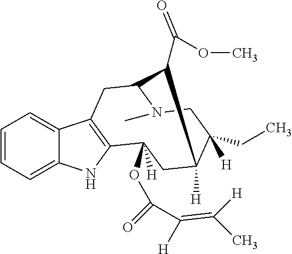 [SMILES: C/C=C/C(=O)O[C@@H]1C[C@@H]2[C@@H](CC)CN(C)C(Cc3c1[nH]c1ccccc31)[C@H]2C(=O)OC] |
| BE1 | C₃₂H₄₂N₂O₄ | 518.32 | 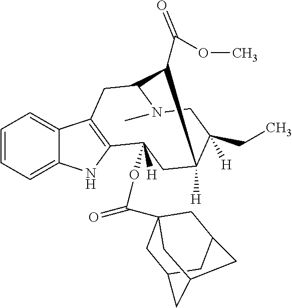 [SMILES: CC[C@@H]1CN(C)C2Cc3c([nH]c4ccccc34)[C@@H](OC(=O)C34CC5CC(CC(C5)C3)C4)C[C@H]1[C@@H]2C(=O)OC] |

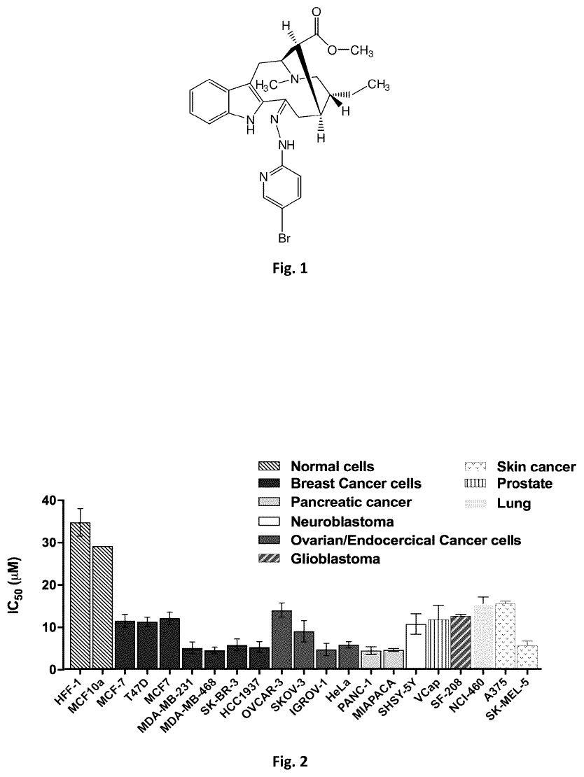

In an embodiment, the compound methyl (5R,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate (COMP; FIG. 1) inhibited the growth of tumor cells expressing different BRCA1/2 status (wild-type, mutant and loss of heterozigoty), but it has a much lower anti-proliferative effect on normal cells (Table2, FIG. 2).

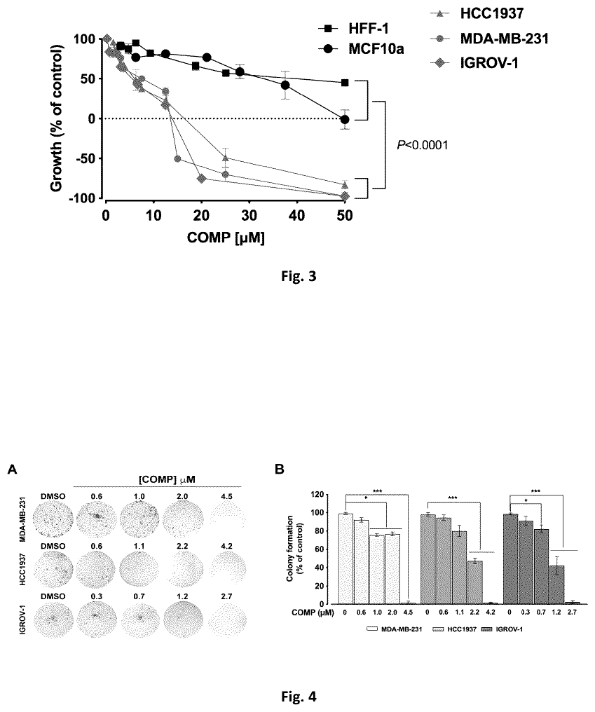

In an embodiment, the activity of COMP compound was tested in an array of human normal and cancer cell lines (Table 2, FIG. 2). The IC₅₀ (concentration of compound that causes 50% growth inhibition) values of the compound ranged from 4.4 μM-12 μM in breast cancer cells (T47D, MCF-7, MDA-MB-231, MDA-MB-468, SK-BR-3 and HCC1937), 4.6-13.9 μM ovarian and endocervical cancer cells (OCVAR, SKOV-3, SK-BR-3, IGROV-1 and HeLa), 4.5 μM in pancreatic cancer cells (PANC-1 and MIAPACA) and 4.5 μM in neuroblastoma cancer cells (SHSY-5Y) (Table 2). The results obtained showed a promising antitumor activity of the compound against distinct types of cancer, including breast (particularly triple-negative breast cancer), ovarian, pancreatic, neuroblastoma, lung, prostate, skin and glioblastoma cancers (Table 2, FIG. 2). Moreover, the IC₅₀ values of the compound are significantly higher in normal human cells, with an IC₅₀ of 29.5 and 33.6 μM in MCF10a and HFF-1, respectively (FIG. 3). Additionally, COMP IC₅₀ values for patient-derived ovarian cancer cells were also assessed (Table 2), ranging from 2.68 μM-15.1 μM.

The effectiveness of COMP against breast and ovarian cancer cells is evidenced when compared to cisplatin (CDDP, clinically used in triple-negative breast cancer and ovarian cancer patients) and olaparib (approved for mutant BRCA1-related breast and ovarian cancers). Moreover, unlike CDDP, the anti-proliferative effect of COMP appears to be highly selective of cancer cells and has an evidently lower effect on normal cells (Table 1). Importantly, COMP is shown to be much more effective than olaparib in all tested cancer cells, regardless of BRCA1 status (Table 2).

Table 2 refers to the growth inhibitory effect of COMP, olaparib and cisplatin (CDDP) in a panel of human immortalized breast (T47D, MCF-7, MDA-MB-231, MDA-MB-468, SK-BR-3 and HCC1937), ovarian and endocervical (OCVAR, SKOV-3, SK-BR-3,IGROV-1 and HeLa), pancreatic (PANC-1 and MIAPACA), neuroblastoma (SHSY-5Y), lung (NCI-H460), prostate (VCaP) melanoma (A375 and SK-MEL-5) and glioblastoma (SF-208) cancer cells, immortalized normal MCF10a and HFF1 human cells, and patient-derived ovarian (PD-OVCA #1, #9, #41, #49 and #62) cancer cells. The half maximal inhibitory concentration (IC50) values were determined by the sulforhodamine B (SRB) assay or CellTiter96®Aqueous one solution cell proliferation (MTS) assay for immortalized or PD-OVCA cells, respectively, after 48 hours of treatment with COMP. Data are mean±SEM (n=5).

TABLE 2
IC₅₀ values obtained for COMP, CDDP, and olaparib in a panel of human immortalized
and patient-derived cancer cells with different BRCA1 and BRCA2 status.

| | | Compounds IC₅₀ (μM) | | |
|---|---|---|---|---|
| Immortalized cancer cells | | COMP | CDDP | Olaparib |
| Wild-type BRCA1/2- | T47D | 11.5 ± 3.0 | 5.9 ± 0.05 | 30.0 ± 2.4 |
| expressing cells | MCF-7 | 12.0 ± 1.5 | 11.9 ± 3.4 | 39.2 ± 2.8 |
| | OVCAR-3 | 13.9 ± 1.7 | 3.2 ± 0.05 | 42.0 ± 4.5 |
| | SKOV-3 | 8.9 ± 2.5 | 12.0 ± 1.1 | 98.0 ± 1.9 |
| | HeLa | 5.8 ± 0.8 | ND | ND |
| | PANC-1 | 4.4 ± 0.9 | ND | ND |
| | MiaPACA | 4.5 ± 0.4 | ND | ND |
| | SHSY-5Y | 10.6 ± 2.4 | 4.5 ± 1.4 | ND |
| | NCI-H460 | 15.5 ± 1.5 | ND | ND |
| | SF-208 | 12.45 ± 0.45 | ND | ND |
| | SK-MEL-5 | 5.65 ± .95 | ND | ND |
| | A375 | 15.5 ± 0.5 | ND | ND |
| | Vcap | 11.7 ± 3.4 | ND | ND |
| BRCA1 loss of | MDA-MB-231 | 5.0 ± 1.4 | 12.1 ± 1.1 | 50.9 ± 1.7 |
| heterozigoty cells | MDA-MB-468 | 4.4 ± 0.8 | 2.3 ± 0.3 | 43.0 ± 5.9 |
| | SK-BR-3 | 5.7 ± 1.4 | 14.5 ± 2.6 | 27.5 ± 3.5 |
| Mutant BRCA1- | HCC1937 (5382insC) | 5.2 ± 1.3 | 8.6 ± 0.9 | 30.5 ± 1.3 |
| expressing cells | | | | |
| Mutant BRCA1/2- | IGROV-1 (mutBRCA1 280delA; | 4.6 ± 1.5 | 5.8 ± 0.7 | 15.9 ± 2.1 |
| expressing cells | mutBRCA2 3320del1) | | | |
| Normal cells | HFF-1 | 33.6 ± 3.2 | 9.3 ± 0.9 | 36.0 ± 1.1 |
| | MCF10a | 29.5 ± 2.7 | 2.3 ± 0.4 | ND |

Patient-derived ovarian (PD-OVCA) cells

| | | | | |
|---|---|---|---|---|
| Pathogenic mutant | PD-OVCA#1 (Lack of | 2.7 ± 0.6 | 15.5 ± 1.4 | 31.9 ± 5.1 |
| BRCA1/2 | exon 17 in BRCA1) | | | |
| | PD-OVCA#9 (mutBRCA1: | 5.1 ± 1.0 | 8.4 ± 1.4 | 15.6 ± 3.5 |
| | c. 4389C > G; | | | |
| | mutBRCA2: c6252delT) | | | |
| Benign mutant BRCA1 | PD-OVCA#41 | 14.1 ± 0.9 | 4.4 ± 1.1 | 64.6 ± 4.4 |
| with pathogenic mut | (mutBRCA2: c. 3707dupA) | | | |
| BRCA2 | | | | |
| Wild-type BRCA1/2- | PD-OVCA#49 | 15.1 ± 1.4 | 8.3 ± 1.8 | 23.6 ± 1.9 |
| expressing cells | PD-OVCA#62 | 14.2 ± 2.9 | 6.5 ± 1.5 | 56.2 ± 5.2 |

*ND: Not determined

In an embodiment, IC₅₀ values were determined by Sulforhodamine B (SRB) or MTS [3-(4,5-dimethylthiazol-2-yl)-5-(3-carboxymethoxyphenyl)-2-(4-sulfophenyl)-2H-tetrazolium] assay in immortalized and PD-OVCA cells, respectively. Cancer cells were plated in 96-well plates and incubated for 24 hours. Cells were then exposed to serial dilutions of compounds for 48 hours. The solvent DMSO corresponding to the maximum concentration used in these assays (0.025%) was included as control. Results are the mean±S.E.M. of 3-5 independent experiments.

In an embodiment, colony-formation assay was performed. The marked inhibitory effect of COMP on triple-negative breast cancer and ovarian cancer cells viability was further demonstrated by colony-formation assay. Once again, BBTI20 significantly reduced the colony-forming ability of cancer cells (FIGS. 4A-B).

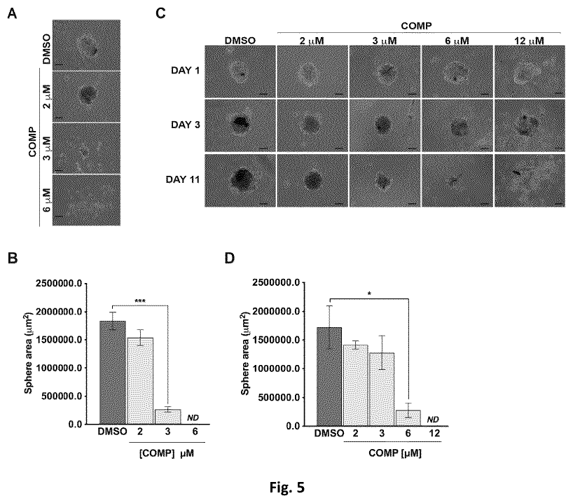

In an embodiment, COMP significantly inhibited mammosphere formation in a 3D-mammosphere model generated from HCC1937 cells, leading to a complete abolishment of spheroids formation at 6 μM, when added upon seeding (FIGS. 5A-B). Moreover, 6 μM and 12 μM of COMP markedly reduced mammosphere growth in three-day old spheroids, triggering mammosphere disintegration at 12 μM (FIGS. 5C-D).

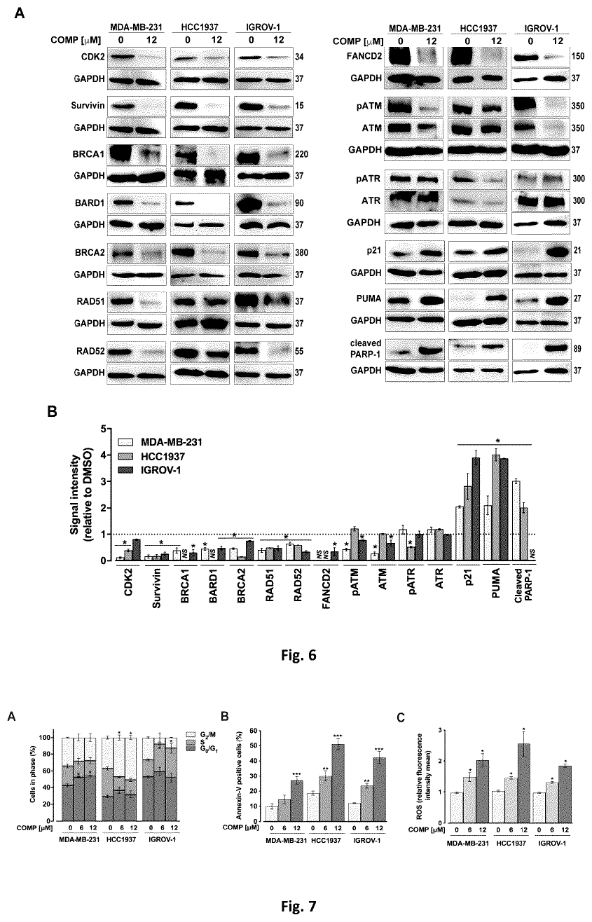

In an embodiment, the COMP compound modulated the expression of key proteins involved in homologous DNA repair, proliferation, chemoresistance, induced cell cycle arrest, apoptosis and ROS generation, in triple-negative breast cancer and ovarian cancer cells. It was shown that 12 μM of COMP significantly decreased the expression levels of proteins associated with DNA damage repair, particularly BRCA1, BRCA2, RAD51, RAD52, FANCD2, pATM, pATR, as well as proteins related to therapeutic resistance (namely CDK2, survivin, BARD1, RAD51 and FAND2; FIGS. 6A-B). Moreover, it was shown that the COMP anti-proliferative effect in triple-negative breast cancer and ovarian cancer cells was associated with the induction of cell cycle arrest at G0/G1- (in MDA-MB-231 cells), S- and G2/M- (in HCC1937 and IGROV-1 cells) phases (FIG. 7A), and increased expression of p21 (FIGS. 6A-B), after 48 hours of treatment.

In an embodiment, COMP-treated cells showed a significant increase in apoptotic cell death, as evident by the increase of PUMA and cleaved PARP protein expression levels (FIGS. 6A-B) and Annexin-V-positive cells (FIG. 7B).

In an embodiment, COMP increases ROS production in COMP-treated cancer cells in a dose-dependent manner (FIG. 7C).

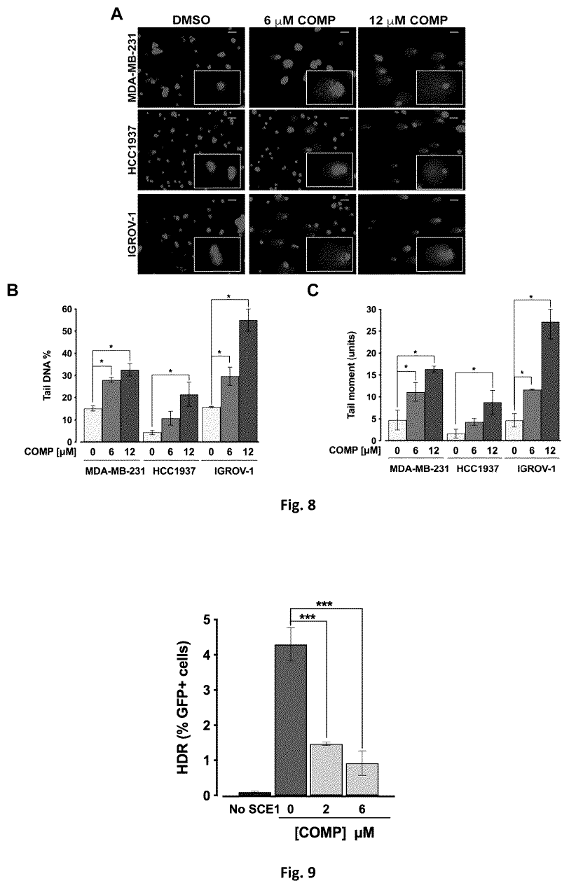

In an embodiment, COMP decreased homologous recombination DNA repair and disrupted the BRCA1-BARD1 interaction. 6 μM and 12 μM of COMP significantly increased the percentage of comet-positive cells, particularly on tail DNA (FIG. 8A and FIGS. 8B) and tail moment (FIG. 8A and FIG. 8C), in MDA-MB-231, HCC1937 and IGROV-1 cells. COMP-treated cells presented a marked reduction in homologous recombination DNA repair capacity, as observed in MCF7 DR-GFP cancer cells treated with 2 μM and 6 μM of COMP on homologous recombination (FIG. 9).

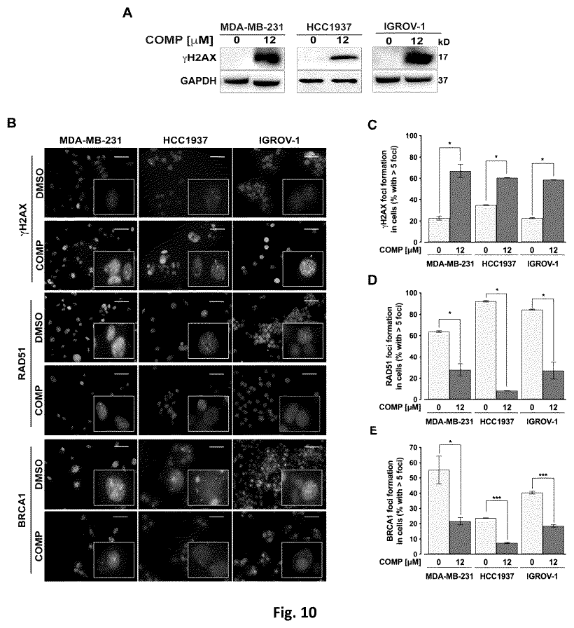

In an embodiment, 12 μM of COMP increased the amount of phosphorylated (Ser139) histone H2AX (γH2AX) (FIG. 10A) and the number of γH2AX-positive foci formed in MDA-MB-231, HCC1937 and IGROV-1 cells (FIG. 10B and FIG. 10C).

In an embodiment, a pronounced reduction in RAD51-foci formation could also be observed by immunofluorescence analysis in COMP-treated cancer cells (FIG. 10B and FIG. 10D).

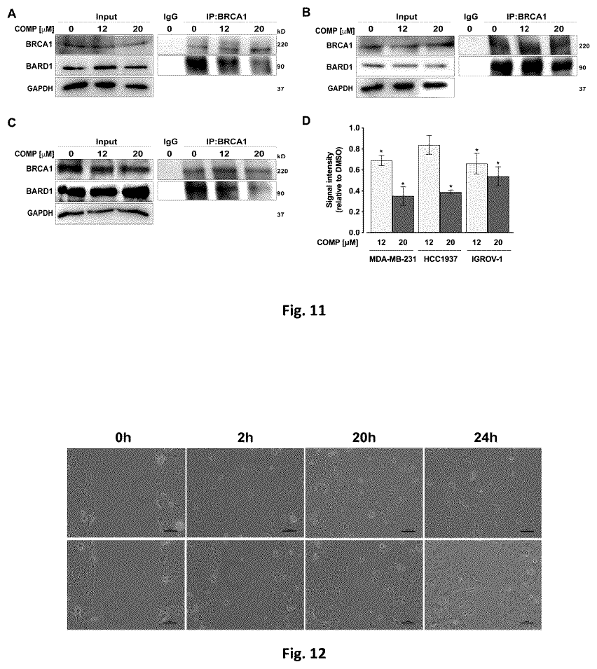

In an embodiment, 12 μM of COMP triggered the nucleocytoplasmic translocation of BRCA1 in MDA-MB-231, HCC1937 and IGROV-1 cells (FIG. 10B and FIG. 10D). This outcome may be due to a disruption of the BRCA1-BARD1 interaction in MDA-MB-231 (FIG. 11A and D), HCC1937 (FIG. 11B and FIG. 11D), and IGROV-1 (FIG. 11C and FIG. 11D) cells, upon treatment with 12 and 20 μM COMP.

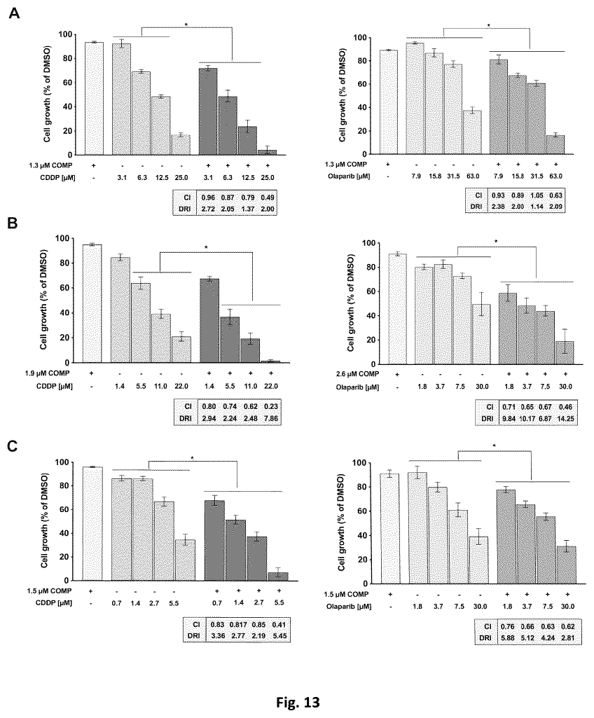

In an embodiment, COMP prevented cell migration of triple-negative breast cancer cells. The effect of COMP on the migration ability of HCC1937 cells was also studied. In the wound healing assay, for 1.9 μM (concentration with no significant effect on cell viability), COMP significantly reduced the wound closure in HCC1937 cells (FIG. 12).

In an embodiment, COMP sensitizes triple-negative breast cancer and ovarian cancer cells to the effect of CDDP and olaparib as shown by the enhancement of the growth inhibitory effect and promising synergistic effects between COMP and CDDP or olaparib (CI<1), with a noticeable reduction in the effective dose of chemotherapeutic agents (FIGS. 13A-C).

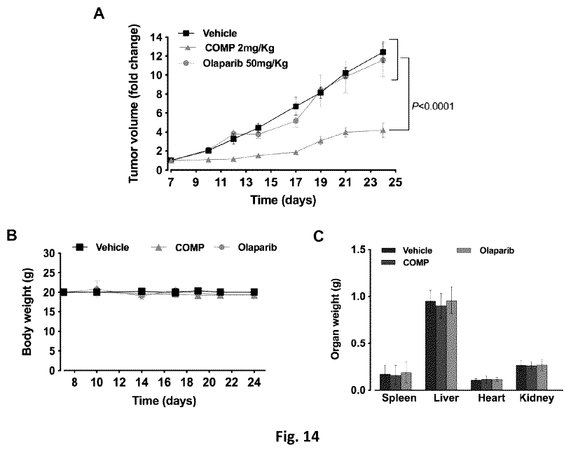

In an embodiment, COMP showed antitumor activity in xenograft mouse models of ovarian cancer cells. In vivo studies using xenograft mice models showed that after seven administrations of 2 mg/kg of COMP, the growth of IGROV-1 tumors was significantly inhibited when compared to vehicle or 50 mg/kg of olaparib administration (FIG. 14A). Additionally, no significant variation of body (FIG. 14B) and organs (FIG. 14C) weight was observed in COMP-treated mice as compared to vehicle.

TABLE 2
IC50 values obtained for COMP, COMP derivatives, CDDP, and olaparib in a panel of human
immortalized and patient-derived cancer cells with different BRCA1 status. The half maximal
inhibitory concentration (IC50) values were determined by the sulforhodamine B (SRB)
assay, after 48 hours of treatment with compounds. Data are mean ± SEM (n = 5).

| | Human cell lines | | | | | | | | | |
|---|---|---|---|---|---|---|---|---|---|---|
| | | | | | BRCA1 Loss of heterozygosity | | | | | Normal |
| | BRCA1 wild-type | | | | MDA- | MDA- | | BRCA1 mutant | | cells |
| | T47D | MCF-7 | OVCAR-3 | Skov-3 | MB-231 | MB-468 | SK-BR-3 | HCC1937 | Igrov-1 | HFF1 |
| COMP | 11.5 ± | 12.0 ± | 13.9 ± | 8.9 ± | 5.0 ± | 4.4 ± | 5.7 ± | 5.2 ± | 4.6 ± | 33.6 ± |
| | 3.0 | 1.5 | 1.7 | 2.5 | 1.4 | 0.8 | 1.4 | 1.3 | 1.5 | 3.2 |
| D | >50 | >50 | >50 | >50 | >50 | >50 | >50 | >50 | >50 | >50 |
| T | >50 | >50 | >50 | >50 | >50 | >50 | >50 | >50 | >50 | >50 |
| DH1 | >50 | >50 | >50 | >50 | >50 | >50 | >50 | 32.1 ± | 26.0 ± | >50 |
| | | | | | | | | 3.1 | 1.0 | |
| DH2 | 23.0 ± | >50 | >50 | 18.8 ± | 23.2 ± | 31.5 ± | 18.0 ± | 19.5 ± | 12.7 ± | 20.5 ± |
| | 1.0 | | | 3.4 | 2.8 | 1.5 | 2.0 | 0.5 | 0.9 | 1.5 |
| DH3 | >50 | >50 | >50 | 27.5 ± | 49.0 ± | 40.0 ± | >50 | >50 | 24.5 ± | 26.5 ± |
| | | | | 1.5 | 1 | 3.0 | | | 2.5 | 1.5 |
| DS2 | 25.5 ± | 39.3 ± | 43.3 ± | 47.5 ± | 31 ± | 33.0 ± | 29.5 ± | 27. ± | 28.5 ± | 45.5 ± |
| | 2.4 | 3.8 | 2.7 | 5.5 | 0.9 | 1.0 | 2.4 | 3.9 | 2.5 | 0.5 |
| DS3 | 27.5 ± | 14.4 ± | 22.2 ± | 28.7 ± | 8.7 ± | 15.5 ± | 20.5 ± | 19.5 ± | 17.5 ± | 18.6 ± |
| | 2.5 | 1.4 | 0.7 | 5.8 | 1.7 | 0.5 | 0.5 | 2.5 | 2.5 | 1.7 |
| TH2 | 22.5 ± | 25.6 ± | 13.5 ± | 23.5 ± | 19.0 ± | 11.5 ± | 14.5 ± | 25.5 ± | 9.7 ± | 19.3 ± |
| | 0.5 | 0.8 | 0.5 | 1.5 | 2.6 | 1.5 | 0.5 | 1.5 | 1.3 | 4.8 |
| TH4 | 23.5 ± | 12.5 ± | 22.0 ± | 14.5 ± | 13.7 ± | 16.0 ± | 16.0 ± | 22.5 ± | 14.0 ± | 16.5 ± |
| | 0.5 | 1.5 | 2.0 | 0.5 | 1.5 | 3.0 | 3.0 | 1.5 | 1.5 | 6.5 |
| DOH | >50 | >50 | >50 | >50 | >50 | >50 | >50 | >50 | >50 | >50 |
| DTHIO | >50 | >50 | >50 | >50 | >50 | >50 | >50 | >50 | >50 | >50 |
| DE1 | >50 | >50 | >50 | >50 | >50 | >50 | >50 | >50 | >50 | >50 |
| BE1 | >50 | >50 | >50 | >50 | >50 | >50 | >50 | >50 | >50 | >50 |
| CDDP | 5.9 ± | 11.9 ± | 3.2 ± | 12.0 ± | 12.1 ± | 2.3 ± | 14.5 + | 8.6 ± | 5.8 ± | 9.3 ± |
| | 0.05 | 3.4 | 0.05 | 1.1 | 1.1 | 0.3 | 2.6 | 0.9 | 0.7 | 0.9 |
| Olaparib | 30.0 ± | 39.2 ± | 42.0 ± | 98.0 ± | 50.9 ± | 43.0 ± | 27.5 + | 30.5 ± | 15.9 ± | 36.0 ± |
| | 2.4 | 2.8 | 4.5 | 1.9 | 1.7 | 5.9 | 3.5 | 1.3 | 2.1 | 1.1 |

Although the preferred embodiments of the present invention have been disclosed for illustrative purposes, those skilled in the art will appreciate that various modifications, additions and substitutions are possible, without departing from the scope of the invention. Therefore, the present invention is not limited to the above-described embodiments, but the present invention is defined by the claims which follow, along with their fall scope of equivalents.

The term “comprising” whenever used in this document is intended to indicate the presence of stated features, integers, steps, components, but not to preclude the presence or addition of one or more other features, integers, steps, components or groups thereof.

It will be appreciated by those of ordinary skill in the art that unless otherwise indicated herein, the particular sequence of steps described is illustrative only and can be varied without departing from the disclosure. Thus, unless otherwise stated the steps described are so unordered meaning that, when possible, the steps can be performed in any convenient or desirable order.

Where singular forms of elements or features are used in the specification of the claims, the plural form is also included, and vice versa, if not specifically excluded. For example, the term “a compound” or “the compound” also includes the plural forms “compounds” or “the compounds,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A method of treating breast cancer, ovarian cancer, endocervical cancer, pancreatic cancer, prostate cancer, skin cancer, lung cancer, glioblastoma, or neuroblastoma in a patient comprising administering to the patient a therapeutically effective amount of a compound of general formula (1), or a pharmaceutically acceptable salt, stereoisomer, diastereoisomer, enantiomer, atropisomer, or polymorph thereof 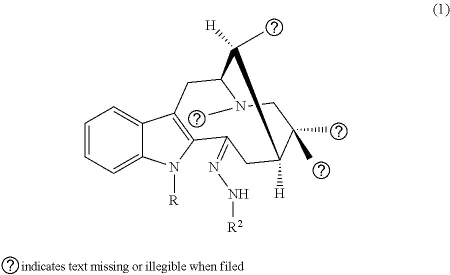
wherein
R¹, R², R³, R⁴, R⁵, R⁶, and R⁷ are independently selected from each other;
R¹ is a H, alkyl, alkenyl, or alkynyl;
R² is an aryl, aroyl, heteroaryl or heteroarylcarbonyl;
R³ is a H or ethyl;
R⁴ is a H or ethyl;
R⁵ is COOR⁶, CH₂OR⁶, CONR⁶R⁷ or CH₂NR⁶R⁷;
R⁶ is a H, alkyl, alkenyl, or alkynyl; and
R⁷ is a H, alkyl, alkenyl, alkynyl, aryl, or heteroaryl.

2. The method according to claim 1, wherein the compound of general formula (1) inhibits the BRCA1 and/or BRCA2 pathway.

3. The method according to claim 1, wherein the compound of general formula (1) inhibits homologous recombination DNA repair through disruption of BRCA1 and/or BRCA2 pathway.

4. The method according to claim 1, wherein the compound of general formula (1) inhibits homologous recombination DNA repair through disruption of BRCA1-BARD1 interaction.

5. (canceled)

6. (canceled)

7. A method of treating triple-negative breast cancer in a patient comprising administering to the patient a therapeutically effective amount of the compound of general formula (1) or a pharmaceutically acceptable salt, stereoisomer, diastereoisomer, enantiomer, atropisomer, or polymorph thereof 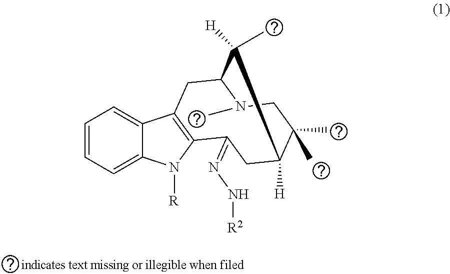
wherein
R¹, R², R³, R⁴, R⁵, R⁶, and R⁷ are independently selected from each other;
R¹ is a H, alkyl, alkenyl, or alkynyl;
R² is an aryl, aroyl, heteroaryl or heteroarylcarbonyl;
R³ is a H or ethyl;
R⁴ is a H or ethyl;
R⁵ is COOR⁶, CH₂OR⁶, CONROR⁷ or CH₂NR⁶R⁷;
R⁶ is a H, alkyl, alkenyl, or alkynyl; and
R⁷ is a H, alkyl, alkenyl, alkynyl, aryl, or heteroaryl.

8. The method of claim 1, with the proviso that methyl(5R,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate and methyl (5,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate are excluded.

9. The method of claim 1, wherein R¹ is a H, C₁-C₆ alkyl, C₁-C₆ alkenyl, or C₁-C₆ alkynyl.

10. The method of claim 1, wherein R² is a heteroaryl.

11. The method of claim 1, wherein R² is a pyridine.

12. The method of claim 1, wherein R² is a pyridine with a substituted halogen.

13. The method of claim 1, wherein R² is 5-bromopyridin.

14. The method of claim 1, wherein R³ is H and R⁴ is ethyl.

15. The method of claim 1, wherein R³ is ethyl and R⁴ is H.

16. (canceled)

17. The method of claim 1, wherein R⁶ is a H, C₁-C₆ alkyl, C₁-C₆ alkenyl, or C₁-C₆ alkynyl.

18. (canceled)

19. The method of claim 1, wherein R⁷ is a C₁-C₆ alkyl, C₁-C₆ alkenyl, or C₁-C₆ alkynyl.

20. The method of claim 1, wherein R⁵ is COOR⁶ and R⁶ is methyl.

21. The method of claim 1, wherein
R¹, R², R³, R⁴, R⁵, R⁶, R⁷, are independently selected from each other;
R¹ is H;
R² is heteroaryl;
R³ is H or ethyl;
R⁴ is H or ethyl;
R⁵ is COOR⁶ or CONROR⁷, R⁶ is H or alkyl;
R⁷ is H, alkyl, alkenyl, alkynyl, aryl, or heteroaryl.

22. (canceled)

23. The method of claim 1, wherein the compound is methyl (5R,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate or methyl (5,6S,14S,E)-8-(2-(5-bromopyridin-2-yl) hydrazineylidene)-5-ethyl-3-methyl-2,3,4,5,6,7,8,9-octahydro-1H-2,6-methanoazecino [5,4-b]indole-14-carboxylate.

24. (canceled)

25. A method of treating breast cancer, ovarian cancer, endocervical cancer, pancreatic cancer, prostate cancer, skin cancer, lung cancer, glioblastoma, or neuroblastoma in a patient comprising administering to the patient a pharmaceutical composition comprising a pharmaceutically effective carrier and a therapeutically effective amount of a compound of general formula (1) or a pharmaceutically acceptable salt, stereoisomer, diastereoisomer, enantiomer, atropisomer, or polymorph thereof 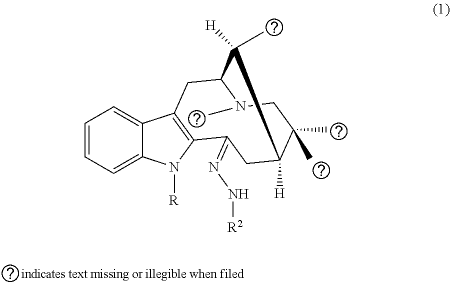
wherein
R¹, R², R³, R⁴, R⁵, R⁶, and R⁷ are independently selected from each other;
R¹ is a H, alkyl, alkenyl, or alkynyl;
R² is an aryl, aroyl, heteroaryl or heteroarylcarbonyl;
R³ is a H or ethyl;
R⁴ is a H or ethyl;
R⁵ is COOR⁶, CH₂OR⁶, CONROR⁷ or CH₂NR⁶R⁷;
R⁶ is a H, alkyl, alkenyl, or alkynyl; and
R⁷ is a H, alkyl, alkenyl, alkynyl, aryl, or heteroaryl.

26. The method of claim 25, wherein the pharmaceutical composition further comprises a chemotherapeutic agent.

27. (canceled)
